(19) United States
(12) Patent Application Publication
(10) Pub. No.: US 2025/0258023 A1
(43) Pub. Date: Aug. 14, 2025

(54) DEFORMABLE SENSOR ARRAYS

(71) Applicant: Tacta Systems Inc., Palo Alto, CA (US)

(72) Inventors: Andreas Bibl, Los Altos, CA (US); Nahid Harjee, Sunnyvale, CA (US); Vikram Pavate, Foster City, CA (US); Patrick M. Smith, San Jose, CA (US); Dariusz Golda, Portola Valley, CA (US)

(21) Appl. No.: 18/775,951

(22) Filed: Jul. 17, 2024

Related U.S. Application Data
(60) Provisional application No. 63/553,095, filed on Feb. 13, 2024.

(51) Int. Cl. G01D 11/30; A61B 5/00

(52) U.S. Cl. CPC G01D 11/30; A61B 5/6807; A61B 5/6812; A61B 5/6893

(57) ABSTRACT

A deformable sensor array may include a plurality of sensors and a flexible substrate. Each sensor of the plurality of sensors may include a sensor die to generate an analog signal and an converter die to perform analog-to-digital conversion (ADC) of the analog signal to generate a digital output. The flexible substrate may include electrical interconnect coupled with the plurality of sensors, and strain relief cutouts between sensors of the plurality of sensors and between the electrical interconnect, to enable deformation of the plurality of sensors. Other aspects are also described and claimed.

### RELATED APPLICATIONS

This patent application claims the benefit of priority of U.S. Provisional Application No. 63/553,095, filed Feb. 13, 2024, which is incorporated herein by reference in its entirety.

### BACKGROUND

### Field

This disclosure relates generally to sensor arrays and, more specifically, to deformable sensor arrays. Other aspects are also described.

### Background Information

A sensor array may refer to a group of sensors used for collecting information about an environment. Sensors of a sensor array may be arranged in a certain geometric configuration or pattern. Sensor arrays may enable collecting information over a greater area than a single sensor, and in two or three dimensions of the environment.

In operation, a sensor of a sensor array can generate an output signal indicating detection of a physical phenomenon. For example, a piezoelectric sensor can utilize the piezoelectric effect and/or the pyroelectric effect to detect changes in pressure, acceleration, temperature, strain, or force by converting such detections to electrical charge. In another example, a capacitive sensor can utilize capacitive sensing to detect an object in proximity that may be conductive or may have a dielectric constant that is different from air.

### SUMMARY

Implementations of this disclosure include a sensing system comprising a deformable sensor array having a plurality of sensors and a flexible substrate. Each sensor of the plurality of sensors may include a sensor die to generate an analog signal and a converter die to perform analog-to-digital conversion (ADC) of the analog signal to generate a digital output. The flexible substrate may be coupled with the deformable sensor array on an upper surface and with an article on a lower surface. For example, the article may be an object (which could also have tight curves and/or a complex shape), such as a glove, sleeve, shoe, seat, dashboard, etc. The flexible substrate may include electrical interconnect coupling sensors of the plurality of sensors, and strain relief cutouts between sensors of the plurality of sensors and between the electrical interconnect. The flexible substrate and the strain relief cutouts may enable deformation of the plurality of sensors with the article.

Some implementations may include a large area (e.g., one square foot or more), multimodal, distributed, deformable sensor array that may be affixed to a complex three-dimensional surface via a flexible substrate, for collecting information about an environment. The sensors may be spread over a large area, such as one square foot or more. The sensor array may be comprised of microfabricated sensor dies bonded to or co-fabricated with local analog-to-digital converter dies to enable digital data to be collected from data bus lines. The digital data can traverse greater distances (e.g., hundreds to thousands of millimeters) without signal degradation, as opposed to analog data which may be attenuated or noisy over such distances. The sensor array may include micro-fabricated sensors (e.g., micro sensors, or sensing pixels) that may have area dimensions in a range, for example, of 100 to 1000 micrometers (μm), and in some cases, 100 μm to 300 μm. This may facilitate large open areas for strain relief cutouts between sensors. The sensors may be spread over the large area at a pitch, for example, >1 mm, and in some cases, >5 mm, or >10 mm, in both X and Y directions (e.g., in a grid pattern). This may enable flexible trace designs of electrical interconnect, coupled with the sensors and a controller, to follow a meandering pattern, such as a serpentine, zigzag, or other shape, to accommodate large deformations of the substrate. Aspects described herein may include local digital conversion of analog sensor signals, and/or large, open areas surrounding the sensors, which enable uniaxial or biaxial stretchability, bendability, and foldability of the sensor array (and flexible substrate) affixed to an article. Other aspects are also described and claimed.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure herein are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1A is an example of a deformable sensor array.

FIG. 1B is an example of strain relief cutouts in a deformable sensor array.

FIG. 2 is a cross-section of an example of a sensor of a deformable sensor array.

FIG. 3 is a cross-section of an example of a deformable sensor array affixed to an article.

FIG. 4 is an example of a deformable sensor array integrated with a seat.

FIG. 5 is an example of a deformable sensor array integrated with a dashboard.

FIG. 6A is an example of a deformable sensor array integrated with a sleeve.

FIG. 6B is an example of a user wearing a deformable sensor array as a sleeve.

FIG. 7A is an example of a deformable sensor array integrated with a shoe.

FIG. 7B is an example of a user wearing a deformable sensor array as a shoe.

FIG. 8 is an example of a deformable sensor array integrated with a sensing glove.

FIG. 9 is an example of a close-up top view of a deformable sensor array.

### DETAILED DESCRIPTION

Large area sensor arrays may be limited by the large, rigid sensors of which they are comprised. These sensors may limit the minimum bending radius of curvature and elongation of the substrate. Large area sensor arrays may also be limited by a sensor fill factor (or sensor density) which limits the overall area available for routing electrical interconnects to and from each of the sensors. They may also be limited by the long wires and connections over which small analog signals, produced by the sensors, may be subject to noise, interference, attenuation, and other signal integrity issues. As a result, applications of sensor arrays are typically limited to environments that do not involve tight curves, complex shapes, and/or high signal-to-noise ratio (SNR).

Implementations of this disclosure address problems such as these by utilizing large area, stretchable, bendable, foldable sensor arrays coupled with flexible substrates (e.g., flexible circuits or backplanes). A microfabricated sensor die (e.g., having a piezoelectric, capacitive, piezoresistive, or temperature sensing area) may be co-located with and connected to an amplifier and/or an ADC of a converter die at each sensor. This may enable digital outputs to be transmitted over greater distances with greater noise-immunity as compared to analog outputs. In this way, digitized sensor signals can be routed over greater distances (e.g., hundreds to thousands of millimeters) in the sensor array, which might otherwise be impractical for low-level analog signals due to noise, attenuation, and/or other signal integrity issues.

The sensors described herein may each include a sensor die coupled with a converter die. The sensor die may include a sensing area. The sensor die can include circuitry to couple with the sensing area and the converter die.

As described herein, for each sensor, amplification and ADC of analog signals may be performed in a microfabricated integrated circuit (IC) converter die. The converter die may have a small size and footprint, such as lateral dimensions that may be in a range of 100 to 1000 μm, and in some cases, in a range of 100 to 300 μm, per side and a thickness in a range of 10 to 100 μm, and in some cases, in a range of 10 μm to 30 μm (e.g., a micro IC). The converter die may include through vias, such as through silicon vias (TSVs), for connecting to the sensor die. The converter die may be coupled with a flexible substrate by conventional pick-and-place mounting methods, such as flip-chip solder bonding. The small footprint of the sensor (via the converter die) may enable a reduced radius of curvature directly under and adjacent to the sensor. The converter die may be coupled with electrical interconnect of the sensor array. The electrical interconnect may include, for example, power, ground, data (e.g., a digital output), clock (e.g., readout synchronization), control (e.g., a configuration input), and/or enable (e.g., a readout trigger). In some cases, the electrical interconnect may include a serial bus.

The microfabricated sensor die may provide an analog signal representing, for example, normal force, shear force, temperature, or proximity. The sensor die may include a sensing area, for sensing the normal force, shear force, temperature, or proximity. The sensor die may have a small size and footprint, such as lateral dimensions in a range of 100 to 1000 μm, and in some cases, in a range of 100 to 300 μm, per side and a thickness in a range of 10 to 100 μm, and in some cases, in a range of 10 to 30 μm (e.g., a micro sensor). Electrical connections of the sensor die may correspond to electrical connections of the converter die, and each sensor die may be bonded to or fabricated with a corresponding converter die (e.g., the sensor die may be in direct electric contact with the converter die). In this way, the converter die may provide power and ground to the sensor die and may amplify and convert analog signals from the sensor die, at the location of the sensor.

By stacking the sensor die relative to the converter die, a greater open area around the sensor may enable mechanical strain reliefs (e.g., cutouts) and meandering patterns of circuit routing (e.g., electrical interconnect). This, in turn, may enable high uniaxial or biaxial stretchability, bendability, foldability, and conformality of the flexible substrate to enable deformation of the sensor array, so that the sensor array can be laminated to complex three-dimensional surfaces of an article to form a sensing system.

In some implementations, the deformable sensor array may be integrated with a seat (e.g., an automotive seat, massage chair, or upholstered seat). The sensor array can then sense information, such as the presence of persons or objects, to provide a closed-loop control of a system, such as applied pressures and/or temperatures of the seat. In some implementations, the sensor array may be integrated with active compression garments to provide a closed-loop control of applied pressure in the active compression garment. The sensor array can sense information to provide a closed-loop control of applied pressures to mitigate, for example, lymphedema, deep vein thrombosis, or varicose veins. In some implementations, the sensor array may be integrated with footwear or compression garments to provide feedback for manual adjustments of the footwear or compression garments, such as to alleviate pressure “hotspots.” In some implementations, the sensor array may be integrated with athletic equipment (e.g., golf grips, shoe insoles) to provide measurements of applied pressure on the equipment, for example, to improve athletic performance. In some implementations, the sensor array may be integrated with large area tactile input surfaces with software-reconfigurable buttons, switches, and/or dials, such as a curved vehicle dashboard, on which control elements can be configured by a user or through over-the-air software updates. In some implementations, the sensor array may be integrated with a mattress (e.g., a hospital bed) to provide pressure mapping, such as to alleviate bed sores. In some implementations, the sensor array may be coupled with a haptic actuator to provide a tactile signal to the user to help locate and interact with the control elements.

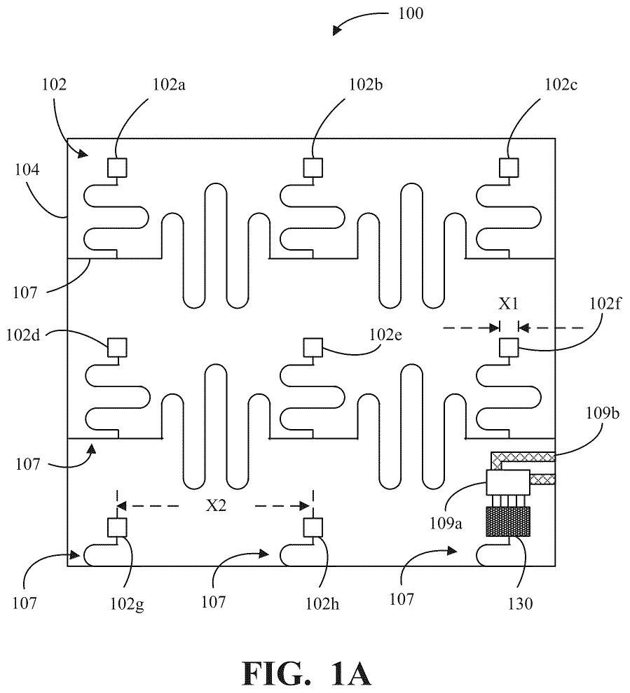

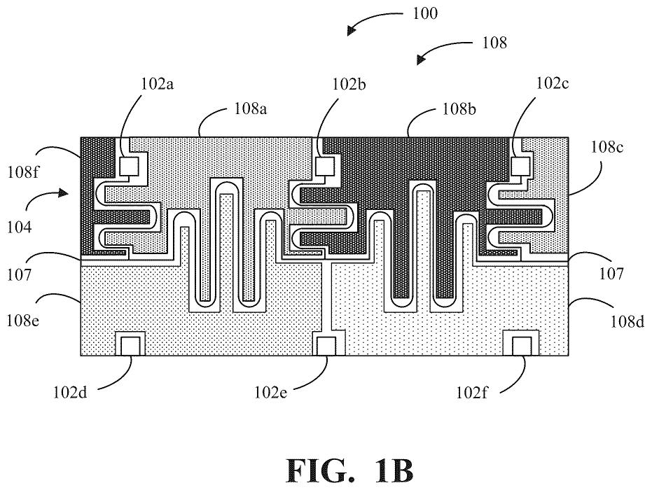

FIG. 1A is an example of a deformable sensor array 100 including a plurality of sensors 102 and a flexible substrate 104. The plurality of sensors 102 may include, for example, sensors 102a to 102h, arranged in a grid. The sensors 102 may be micro sensors spread over a large area, such as one square foot or more. Each sensor 102 may include a sensor die to generate an analog signal and a converter die to perform amplification and ADC of the analog signal to generate a digital output. The sensor die and the converter die may be stacked relative to one another as described herein. In some cases, the sensor array 100 may provide multimodal sensing by having different sensors 102 to sense combinations of normal forces, shear forces, temperatures, and/or proximities (e.g., sensor 102a sensing a normal force, sensor 102b sensing a shear force, sensor 102c sensing a temperature, sensor 102d sensing a proximity, and so forth). The plurality of sensors 102 may be coupled with the flexible substrate 104 which, in turn, may be coupled with or affixed to an article to be used by a user such as via lamination and/or an adhesive.

The sensor array 100 may include, for example, 1,000 sensors, 10,000 sensors, or more, integrated with the article. In some cases, the article may have a plurality of sensor arrays 100, and each sensor array 100 may be utilized based on its location on the article. For example, each sensor 102 of the array may have relatively smaller lateral dimensions (X1), such as, in a range of 100 to 1000 μm, and in some cases, 100 μm to 300 μm. This may facilitate large open areas for strain relief cutouts 108 between sensors 102 (discussed with respect to FIG. 1B). The sensors of the array may also be spread over a relatively larger area with pitch (X2) (as compared to the smaller lateral dimensions (X1), for example, >1 mm, and in some cases, >5 mm, or >10 mm, in both X and Y directions (e.g., in a grid pattern).

The flexible substrate 104 may include electrical interconnect 107 coupled with the sensors 102. The electrical interconnect 107 may include a common set of signal wires or connections for controlling digital readout of the sensors 102. For example, the electrical interconnect 107 may include connections such as V_{DD} (power), V_{SS} (ground), clock, select (e.g., digital input (e.g., to trigger a readout of a sensor 102), and/or digital output (e.g., 8 bits, 12 bits, or more, representing sensing performed by the sensor at a given time). In some cases, the electrical interconnect 107 may include connections to implement a serial bus, such as an inter-integrated circuit (I²C) bus, a SPI bus, a differential signaling bus, or a system management (SM) bus. The electrical interconnect 107 may have a meandering pattern of circuit routing between the sensors 102, and a controller 130 coupled with the sensors 102.

With additional reference to FIG. 1B, the flexible substrate 104 may include a plurality of strain relief cutouts 108 between the sensors 102 and between the electrical interconnect 107 in X and Y directions, such as strain relief cutouts 108a to 108f. For example, the flexible substrate 104 may include strain relief cutout 108a between sensors 102a and 102b, and between legs of a serpentine routing of the electrical interconnect 107 of those sensors. The strain relief cutouts 108 may enable high uniaxial or biaxial stretchability, bendability, foldability, and conformality of the sensor array 100.

Further, the strain relief cutouts 108 may be between sensors 102 in different directions. For example, strain relief cutout 108a may be between sensors 102a and 102b in a first direction (horizontally, along an X-axis in the X direction) and between sensors 102a and 102d in a second direction (vertically, along a Y-axis in the Y direction). As a result, the sensor array 100 may enable a reduced radius of curvature that, in turn, enables coupling with tight contours of an article, such as a wearable device (e.g., a glove, sleeve, or shoe) or other system providing localized control (e.g., a seat or dashboard).

Referring again to FIG. 1A, the controller 130 may perform a fast digital readout of the sensors 102. For example, the controller 130 can cause a pulse to be sent to a digital input of a first sensor, e.g., sensor 102a, to trigger a measurement from that sensor. After that measurement is performed, and data from the sensor is transmitted back to the controller 130, the first sensor can then trigger a second sensor to perform a measurement and transmit data, e.g., sensor 102b, and so forth. For example, the sensor array 100 may include a daisy chain connection of sensors 102 providing serial readout. In some cases, the controller 130 can selectively trigger one or more sensors 102 via a serial bus interface, such as by utilizing a clock and bi-directional digital data. As a result, the controller 130 can selectively cause digital inputs to be transmitted to sensors 102, then read digital outputs from the sensors 102, to perform the readout and receive the measurements.

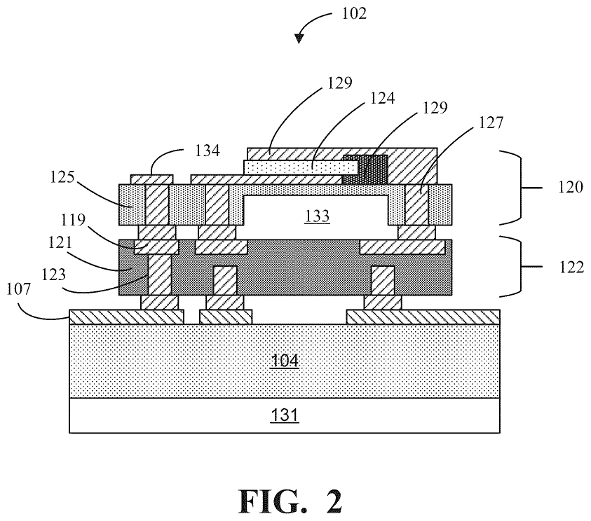

With additional reference to FIG. 2, each sensor 102 may include a sensor die 120 coupled with a converter die 122 (e.g., each a microfabricated IC). In various configurations, the sensor die 120 could perform force, temperature, or proximity sensing. The sensor die 120 may include a microfabricated sensing area 124 for sensing a normal force, shear force, temperature, and/or proximity. In some implementations, the sensing area 124 may comprise a piezoelectric, piezoresistive, capacitive, or other sensing structure. The sensor die 120 may implement circuitry coupled with the sensing area 124 on a first side and with the converter die 122 on a second side. For example, the sensor die 120 could implement circuitry to connect the sensing area 124 to the converter die 122. The sensor die 120 may generate an analog output based on sensing (e.g., corresponding to the article touching a user or object), and the sensor die 120 can transmit the analog output to the converter die 122. A cavity 133 may optionally be formed in the base substrate 125 of the sensor die 120 to enable flex of the sensor die 120 with an application of force directed to the sensing area 124. This may further improve sensitivity and/or deformability.

In the exemplary implementation illustrated, the sensor die 120 can include a base substrate 125. Where the circuitry is included, the base substrate 125 may be silicon or a III-V semiconductor, for example, with the circuitry and back-end-of-the-line (BEOL) routing 134 formed using customary techniques. As shown, the BEOL routing 134 can include landing pads, for example, for external connection, as well as routing for connection with the sensing area 124 and through vias 127. As shown, a plurality of through vias 127 can extend through the base substrate 125 of the sensor die 120 to provide vertical interconnection to the converter die 122. In a particular implementation, the through vias 127 can be TSVs where the base substrate 125 is silicon. A plurality of leads 129 may be further connected with the sensing area 124, and electrically connected with the working circuitry of the sensor die 120 and/or the through vias 127.

The sensor die 120 may provide an analog signal indicating a measurement from sensing, such as a measurement of a normal force, shear force, temperature, or proximity of an object, detected via the sensing area 124. The sensor die 120 may be relatively small, having lateral dimensions, for example, in a range of 100 to 1000 μm, and in some cases, 100 to 300 μm, per side, and a thickness in a range of 10 to 100 μm, and in some cases, 10 to 30 μm. Bottom side electrical connections of the sensor die 120 may be connected to (e.g., bonded to) corresponding electrical connections on a top side of the converter die 122, for example, with solder bumps, metal-metal bonds, or other suitable electrically conductive bonding material. The sensor die 120 may be bonded to or fabricated with the converter die 122 (e.g., in direct electric contact) with the sensing area 124 exposed on a top of the sensor 102. In this way, the converter die 122 may provide power and ground to the sensor die 120 and may amplify and convert analog signals from the sensor die 120 at the exact location of the sensor 102 in the sensor array 100.

Bonding between the sensor die 120 and the converter die 122 could be performed, for example, at the die level with a pick-and-place process, or at the wafer level followed by singulation. Stacking the sensor die 120 on the converter die 122 for a given sensor 102 can facilitate integration of a greater number of sensors 102 per unit area in the sensing system. Additionally, this may enable more available area for strain relief cutouts 108 to allow greater deformability of the article 150.

The converter die 122 may include circuitry to perform amplification and ADC of the analog output, from the sensor die 120, to generate a digital output that is a representation of the analog output. For example, the converter die 122 could implement a charge amplifier to amplify voltages and/or currents, generated by the sensor die 120, for the ADC. The digital output could comprise 8 bits, 12 bits, or more, representing the analog sensing.

In the exemplary implementation illustrated, the converter die 122 can include a base substrate 121. Where the circuitry is included, the base substrate 121 may be silicon or a III-V semiconductor, for example, with the circuitry and BEOL 119 formed using customary techniques. As shown through vias 123 can extend through the base substrate 121 of the converter die 122 to provide vertical interconnection between the sensor die 120 and electrical interconnects 107 (e.g., copper wiring) over the flexible substrate 104. This can optionally be supported by a substrate 131 (e.g., glass, polymer) that can be adhered to the article 150 on a bottom side. In a particular implementation, the through vias 123 can be TSVs where the base substrate 121 is silicon.

The converter die 122 could be a MOSFET or CMOS IC comprised of single-crystalline silicon. Like the sensor die 120, the converter die 122 may have lateral dimensions, for example, in a range of 100 to 1000 μm, and in some cases, 100 to 300 μm, per side, and a thickness in a range of 10 to 100 μm, and in some cases, 10 to 30 μm.

In some implementations, the converter die 122 may be coupled with the flexible substrate 104 by conventional pick-and-place mounting methods (e.g., flip-chip solder bonding). The sensor die 120 and the converter die 122 may be micro-fabricated separately from the flexible substrate 104 and subsequently assembled to the flexible substrate 104 (which may be coupled with the article 150). In some implementations, each sensor array 100 may be coupled with its own flexible substrate 104.

The small footprint of the sensor die 120 and the converter die 122 may enable a small radius of curvature directly under and adjacent to the sensor 102. The converter die 122 may be coupled with electrical interconnect 107 (e.g., copper wiring) of the sensor array 100 that may, in turn, be coupled with other components of the sensing system (e.g., other sensors 102 and/or the controller 130).

Referring again to FIG. 1A, the controller 130 (e.g., another IC in the sensing system, such as an application-specific integrated circuit (ASIC), microcontroller, or field-programmable gate array (FPGA)) may be connected to the plurality of sensors 102. The controller 130 may also connect to a communications device 109a which, in some cases, may include an antenna 109b to enable wireless communications with another system. The antenna 109b could be a microstrip antenna coupled with the flexible substrate 104, along with the communications device 109a and the controller 130. In some implementations, the communications device 109a may enable wired communications with the other system, without an antenna.

In operation, the controller 130 can cause one or more sensors 102 to each transmit a digital output. A digital output from a sensor 102 may indicate sensing performed by that sensor 102 at a given time in response to receiving the digital input (e.g., a trigger to cause a readout), and might also contain stored readings. In some cases, the controller 130 can directly cause transmission of a digital output from a sensor 102, such as by sending a digital input to the sensor 102. In other cases, the controller 130 can indirectly cause transmission of a digital output from a sensor 102, such as by causing a local controller to send a digital input to the sensor 102, and/or by causing one sensor 102 to send a digital input to trigger another sensor 102 (e.g., sensors connected in a daisy chain).

The communications device 109a may enable transmission of a collection of digital outputs from sensors 102 to another system. The communications device 109a may utilize wired or wireless connections, such as universal serial bus (USB), low-voltage differential signaling (LVDS), serial peripheral interface (SPI), Bluetooth, or Ethernet, to transmit the digital data. For example, the controller 130 can receive digital outputs from the sensors 102 based on triggering those sensors, then utilize the communications device 109a to transmit a compressed digital bitstream encoding the digital outputs to another system, such as a host computer or server, via the antenna 109b. As a result, the controller 130 can selectively perform readout of sensors 102 of the sensor array 100 to obtain sensing information with high temporal resolution while the sensor array is deforming or is deformed with the article.

In some implementations, a sensing system may include a plurality of sensor arrays 100. Each sensor array 100 may be coupled with its own flexible substrate 104. For example, the controller 130 of each sensor array 100 could be a local controller connected to a global controller in the sensing system.

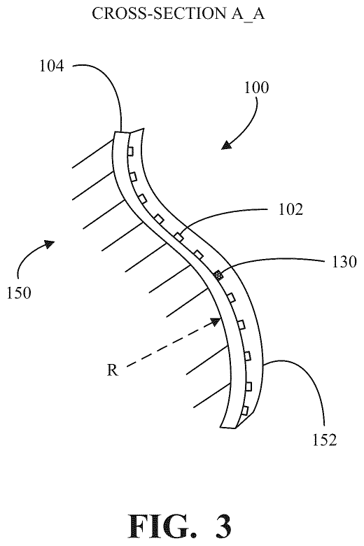

FIG. 3 is an example of a cross-section of a sensor array 100. The plurality of sensors 102, coupled with the flexible substrate 104, may be shaped to conform to contours of an article 150 to which the sensor array 100 is applied or affixed. The article 150 may have tight curves and/or a complex shape, such as a glove, sleeve, shoe, seat, dashboard, etc. For example, the sensor array 100 could be shaped to conform to contours of an active compression garment to provide a closed-loop control (e.g., via the controller 130) of applied pressure in the active compression garment, such as to mitigate lymphedema, deep vein thrombosis, or varicose veins. In another example, the sensor array 100 may be shaped to conform to contours of footwear or compression garments to provide feedback (e.g., via the controller 130) for manual adjustments of the footwear or compression garments, such as to alleviate pressure “hotspots.” In another example, the sensor array 100 may be shaped to conform to contours of athletic equipment (e.g., golf grips, shoe insoles) to provide measurements of applied pressures (e.g., via the controller 130) on the equipment, for example, to improve performance of an athlete.

The flexible substrate 104 may be adhered to the contours of the article 150, such as by lamination and/or adhesive. Further, the sensors 102 may be encapsulated in an encapsulation layer 152 (e.g., silicone) to protect the sensor array 100 from environmental conditions while sensing. The small footprint of the sensors 102 may enable a reduced radius of curvature (shown as “R” in FIG. 3) directly under and adjacent to the sensors 102 to enable the stretching, bending, folding, and conforming of the sensor array 100.

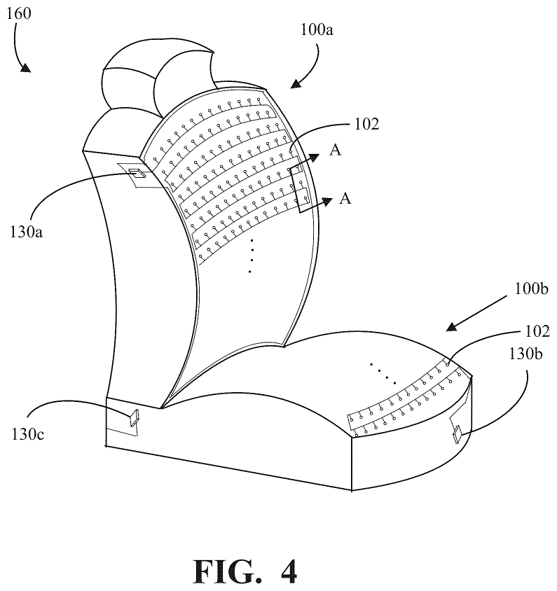

FIG. 4 is an example of a sensing system including a plurality of sensor arrays integrated with an article, such as a seat 160 (e.g., the article 150, shown in cross-section A-A of FIG. 3). For example, the seat 160 could be an automotive seat, massage chair, or other furnishing. The plurality of sensor arrays may include sensor arrays 100a and 100b integrated with, or affixed to, different portions of the seat 160. For example, the flexible substrates 104 of the sensor arrays may each be coupled with upholstery of different portions of the seat 160, such as sensor array 100a coupled with a back portion, and sensor array 100b coupled with a seat portion. The plurality of sensors 102, coupled with the flexible substrates 104 of the different sensor arrays, may stretch, bend, and fold to follow contours of the seat 160 and may be deformable with persons and objects in the seat 160. The sensor arrays can sense information, such as whether an occupant or object is in the seat 160 and/or a weight of the occupant or object. Local controllers of the sensor arrays, such as local controllers 130a and 130b, can communicate with a global controller of the sensing system, such as global controller 130c. The global controller 130c can receive sensing input and utilize closed-loop control, for example, to regulate one or more aspects of a system, such as pressures and/or temperatures applied by the seat 160, based on the sensed conditions indicated by the sensor arrays.

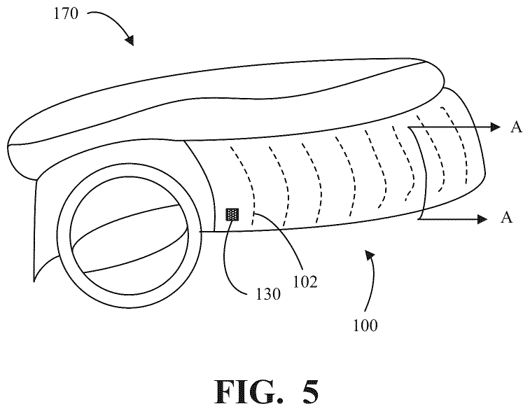

FIG. 5 is an example of a sensing system including a sensor array 100 integrated with an article, such as a dashboard 170 (e.g., the article 150, shown in cross-section A-A of FIG. 3) of an automobile or vehicle. The dashboard 170 may represent a large, curved, contoured area (e.g., a square foot or more) with complex three-dimensional surfaces. The sensor array 100 may provide tactile input surfaces for a user, and in some cases, may include software-reconfigurable buttons, switches, and/or dials. The sensor array 100 can sense information from the user and provide closed-loop control (via the controller 130) to configure and control elements of the dashboard 170, for example by reconfiguring buttons for a driving mode vs. a parked mode.

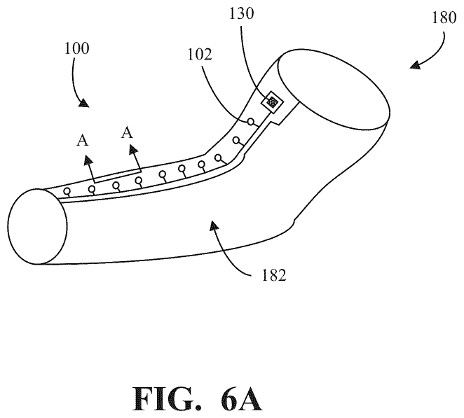

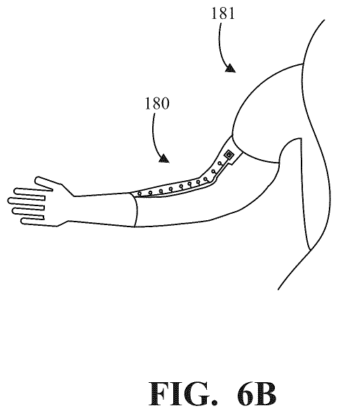

FIG. 6A is an example of a sensing system including a sensor array 100 integrated with an article, such as a sleeve 180 (e.g., the article 150, shown in cross-section A-A of FIG. 3). The sleeve 180 may be worn, for example, to provide compression to an arm (or leg) of a user 181 (or worn on a robotic arm or leg) as shown in FIG. 6B. For example, the flexible substrate 104 may be coupled with an elastic textile surface 182 of the sleeve 180. The plurality of sensors 102, coupled with the flexible substrate 104, may stretch, bend, and fold to follow contours of the sleeve 180, and may be deformable with motion of the user's arm or leg or a robotic arm or leg. For example, the sensor array 100 can sense information of the wearer of the sleeve 180 and provide a closed-loop control (via the controller 130) of pressures applied by the sleeve 180 to the wearer.

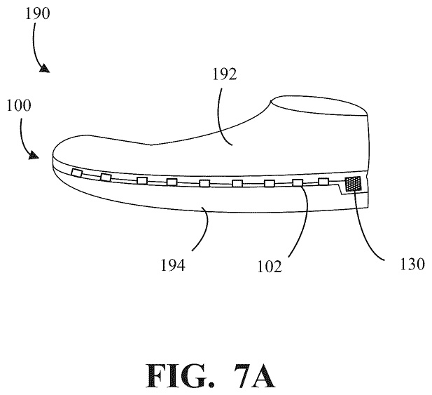

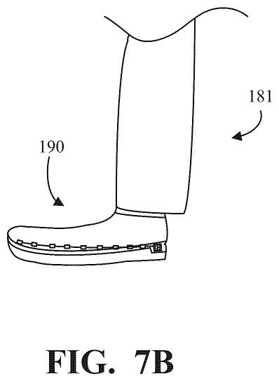

FIG. 7A is an example of a sensing system including a sensor array 100 integrated with an article, such as a shoe 190 (e.g., an athletic shoe). The shoe 190 may be worn on a foot of the user 181 (or worn on a robotic foot) as shown in FIG. 7B. For example, the shoe 190 could be the article 150, shown in cross-section A-A of FIG. 3. The sensor array 100 may be integrated, or affixed, between an upper portion 192 of the shoe 190 and a lower portion or sole 194 of the shoe 190. For example, the encapsulation layer 152 shown in FIG. 3 may be coupled with the upper portion 192, and the flexible substrate 104 may be coupled with the lower portion or sole 194, with the sensor array 100 in between (e.g., configured as a midsole in this example). The plurality of sensors 102, coupled with the flexible substrate 104 and the encapsulation layer 152, may stretch, bend, and fold to follow contours of the shoe 190 and may be deformable with motion of the user or robotic device (e.g., stepping or jumping).

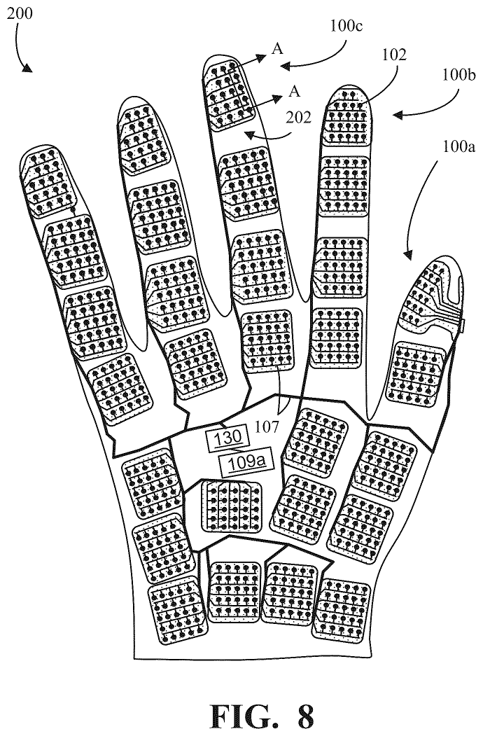

FIG. 8 is an example of a sensing system including a plurality of sensor arrays 100 integrated with an article (e.g., the article 150, shown in cross-section A-A of FIG. 3). For example, the article may be a sensing glove 200 worn on a hand of a user or a robotic hand. The sensor arrays 100 may be woven or adhered to an exterior surface 202 of the sensing glove 200. The sensor arrays 100 may be integrated with different portions of the sensing glove 200, such as fingers, thumbs, and/or palm locations on a palmar side of the glove. For example, a first sensor array 100a may correspond to a first group of sensors 102 (e.g., 10 sensors, 100 sensors, or more) arranged on a thumb or thumb tip, a second sensor array 100b may correspond to a second group of sensors 102 (e.g., another 10 sensors, 100 sensors, or more) arranged on a first finger or fingertip, a third sensor array 100c may correspond to a third group of sensors 102 (e.g., another 10 sensors, 100 sensors, or more) arranged on a third finger or fingertip, and so forth. Sensor arrays 100 may also be arranged between joints of fingers/thumbs and/or contours of the palm.

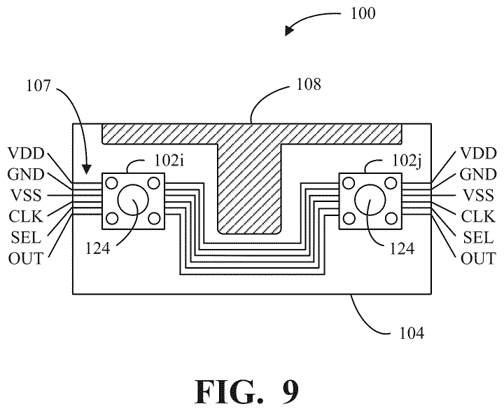

A plurality of flexible substrates 104 may be coupled with the exterior surface 202 (e.g., a textile surface) for each sensor array 100. The sensor arrays 100, coupled with the flexible substrates 104, may stretch, bend, and fold to follow contours of the sensing glove 200 and may be deformable with motion of the user or robotic hand. For example, the sensor arrays 100 can sense information of the wearer of the sensing glove 200. FIG. 9 is an example of a close-up top view of a sensor array 100, including electrical connections between sensors 102i and 102j, via electrical interconnect 107, shown by way of example. The sensors 102 may be connected in series (e.g., a daisy chain) or in parallel via one or more signals of the electrical interconnect 107, such as a select line (“SEL”) or digital input. The number of electrical signals connected to the sensors 102 can be reduced by the sensors 102 sharing a same basic, common set of signal wires, such as power 1 (“V_{DD},” a high voltages supply), ground (“GND”), clock (“CLK”), a select line (“SEL”), a digital output (“OUT”), and/or power 2 (“V_{SS},” a low voltage supply). The controller 130 can utilize the select line to send a digital input to the sensors 102 and utilize the digital output to receive outputs from the sensors 102. In some cases, the electrical interconnect 107 may include a serial bus, such as an I²C bus, a SPI bus, or an SM bus. The controller 130 can utilize the clock to synchronize the outputs from the sensors 102 during the readout. In some cases, additional signals may be used for further control and/or synchronization of the read out.

As discussed above with respect to FIG. 1B, the flexible substrate 104 may include strain relief cutouts 108 between the sensors 102 and the electrical interconnect 107. For example, the flexible substrate 104 may include strain relief cutouts 108 between sensors 102i and 102j and their electrical connections. The strain relief cutouts 108 may be a physical absence of material in the flexible substrate 104, or a softer material, to accommodate flex, fold, and strain of the article when worn by a user or a robotic device.

As used herein, the term “circuitry” refers to an arrangement of electronic components (e.g., transistors, resistors, capacitors, and/or inductors) that is structured to implement one or more functions. For example, a circuit may include one or more transistors interconnected to form logic gates that collectively implement a logical function.

In utilizing the various aspects of the embodiments, it would become apparent to one skilled in the art that combinations or variations of the above embodiments are possible for utilizing deformable sensor arrays. Although the embodiments have been described in language specific to structural features and/or methodological acts, it is to be understood that the appended claims are not necessarily limited to the specific features or acts described. The specific features and acts disclosed are instead to be understood as embodiments of the claims useful for illustration.

What is claimed is:

1. A deformable sensor array, comprising:
a plurality of sensors, each sensor including a sensor die to generate an analog signal and a converter die to perform analog-to-digital conversion (ADC) of the analog signal to generate a digital output; and
a flexible substrate coupled with the plurality of sensors, the flexible substrate including electrical interconnect coupled with the plurality of sensors, and strain relief cutouts between sensors of the plurality of sensors and between the electrical interconnect, to enable deformation of the plurality of sensors.

2. The deformable sensor array of claim 1, wherein the sensor die comprises a sensing area to sense at least one of a normal force, shear force, temperature, or proximity.

3. The deformable sensor array of claim 1, wherein the sensor die is stacked above the converter die, and wherein the converter die is stacked above the flexible substrate.

4. The deformable sensor array of claim 1, wherein the sensor die is bonded to the converter die, and wherein the converter die is bonded to the electrical interconnect.

5. The deformable sensor array of claim 1, wherein the converter die includes through vias to couple with the sensor die on a top side and with the flexible substrate on a bottom side.

6. The deformable sensor array of claim 1, wherein the flexible substrate is coupled with an article, to be used by a user, via at least one of lamination or adhesive.

7. The deformable sensor array of claim 1, wherein the strain relief cutouts are between a serpentine routing of the electrical interconnect.

8. The deformable sensor array of claim 1, wherein at least one strain relief cutout is between a first set of sensors in a first direction and between a second set of sensors in a second direction.

9. The deformable sensor array of claim 1, wherein the deformable sensor array is multimodal with sensors that sense two or more of a normal force, shear force, temperature, or proximity.

10. The deformable sensor array of claim 1, further comprising:
a controller coupled with the plurality of sensors and coupled with the flexible substrate, wherein the controller triggers one or more sensors of the plurality of sensors to generate the digital output.

11. The deformable sensor array of claim 1, further comprising:
a communications device coupled with a controller and with the flexible substrate, wherein the communications device transmits a digital bitstream representing digital outputs from the plurality of sensors.

12. The deformable sensor array of claim 1, wherein the electrical interconnect comprises a serial bus connected to the plurality of sensors.

13. The deformable sensor array of claim 1, wherein each sensor of the plurality of sensors has a lateral dimension that is less than 1000 μm, and wherein the deformable sensor array includes sensors spread over at least one square foot with a thickness less than 100 μm.

14. A sensing system, comprising:
an article to be used by a user;
a flexible substrate coupled with the article; and
a plurality of sensors coupled with the flexible substrate, each sensor of the plurality of sensors including a sensor die to generate an analog signal and a converter die to perform analog-to-digital conversion (ADC) of the analog signal to generate a digital output,
wherein the flexible substrate includes electrical interconnect coupled with the plurality of sensors, and strain relief cutouts between sensors of the plurality of sensors and between the electrical interconnect, to enable deformation of the plurality of sensors with contours of the article.

15. The sensing system of claim 14, further comprising:
a controller in communication with the plurality of sensors, wherein the controller provides closed-loop control based on a sensed condition indicated by the plurality of sensors.

16. The sensing system of claim 14, further comprising:
a global controller in communication with a first local controller coupled with the plurality of sensors and the flexible substrate and a second local controller coupled with a second plurality of sensors and a second flexible substrate.

17. The sensing system of claim 14, wherein the article is a seat and the flexible substrate is coupled with upholstery of the seat.

18. The sensing system of claim 14, wherein the article is a dashboard and the flexible substrate is coupled with a curved contour of the dashboard.

19. The sensing system of claim 14, wherein the article is a compression sleeve and the flexible substrate is coupled with an elastic textile surface of the compression sleeve.

20. The sensing system of claim 14, wherein the article is a shoe and the flexible substrate is arranged between an upper portion and a sole of the shoe.
